(19) United States
(12) Patent Application Publication
(10) Pub. No.: US 2002/0136756 A1
(43) Pub. Date: Sep. 26, 2002

(54) Method and article for debridement and detoxification of wounds

(76) Inventor: John B. McAdams, (US)

Correspondence Address: Akerman, Senterfitt & Eidson, P.A., Post Office Box 3188, West Palm Beach, FL, 33402-3188, US

(21) Appl. No.: 09/940,007

(22) Filed: Aug. 27, 2001

Related U.S. Application Data
(63) Continuation of application No. 09/222,111, filed on Dec. 29, 1998, now abandoned.

(51) Int. Cl. A61F 13/00

(52) U.S. Cl. 424/443; 424/400

(57) ABSTRACT

A method for delivering an sub-epidermal treatment by preparing a medicating solution and saturating a stauratable material with sufficient abrasive characteristics to abrade the epidermis and abrading said epidermis where a said abrasion enables sub-epidermal delivery of said medicating solution in said saturated material.

### CROSS-REFERENCE TO RELATED APPLICATIONS

This application is a continuation of my co-pending U.S. patent application Ser. No. 09/222,111, filed Dec, 29, 1998.

### TECHNICAL FIELD

The invention relates to a method to treat bites, stings or wounds cause by fire ants, sea lice, related arthropod and cnidarians, as well as other biological wounds and envenomations.

### BACKGROUND OF THE INVENTION

Fire ants (Solenopsis), sea lice (one of 1000 species of Cnidarians), and related arthropod and cnidarians have in many ways has made tropical paradises the bane of many human visitors from colder climates to apparent paradise. Of particular interest is the fire ant or Solenopsis Invicta or S. Invicta that was inadvertently introduced to the United states by a freighter in Texas that had arrived from South America. There were some species of Solenopsis were indigenous to the United States prior to the arrival of Solenopsis Invicta. The arrival of S. Invicta or fire ants has caused great concern because the fire ants' aggressive behavior, expanding migration throughout the North American, ruggedness, and especially because of the highly painful, toxic and morbid stings of these little creatures. Not only is the sting of the S. Invicta highly painfully, in some cases it has been fatal to the recipient. Since S. Invicta forages in packs, seldom does a victim receive just a single sting. S. Invicta release an airborne chemical that acts as a message that one is in the attack mode and this triggers all nearby S. Invicta to begin the attack on the same prey. The debilitating effect of such a massive attacks, and overwhelming lighting assault oftentimes causes smaller creatures to become incapacitated, leaving them as easy prey for the entire nest S. Invicta.

Although introduced in Texas 50 years ago, S. Invicta or the fire ant, now ranges from Virginia to California and most states of the Union and Countries of the World south of that latitude. Humans are not the only victims of S. Invicta, but all creatures have been known to fall prey. Juveniles and particularly newborns are especially vulnerable to Solenopsis Invicta. In Florida, one of the numerous dangers for sea turtles is S. Invicta because they attack the turtle's nest, eggs and newly hatched young with zeal.

Despite the horrendously painful sting, which endures for hours, what is even worse is the pustule formation. The pustule, which resembles a small boil or a large white head pimple, invites rupture either intentionally or accidentally because of its raised distended presence. Once rupture, those open sores are susceptible to infection particularly because they are usually rubbed or scratched by the recipient who is irritated by these repulsive pustules and subsequent sores. The infection of these sores almost always results in some form of scarring to the recipient.

Problems with all cnidarians sea lice (incorrectly called that by the public and the named has stuck) or the larval stage of the thimble jellyfish (Linuche Unguiculata) are as acute, but the fire ant envenomation occurs more often probably because both fire ant and the human are land based creatures.

The treatment of the current invention is directed in this instances to deal with fire ant, sea lice and related arthropod and cnidarians, as well as all other similar biological intoxications. Current treatments for envenomation by fire ants and cnidarians are similar and done as much for the placebo effect as for true efficacy, especially topical treatments. Often the treatments of choice are topical measures such as the application of a cortisone cream, colloidal preparation, or the omnipresent calamine solution. In more severe cases, antihistamines, steroids, and epinephrine may be administered by I.M., I.V., and/ or orally. In all cases the recipient is told not to scratch or abrade the wound in any way.

### SUMMARY OF THE INVENTION

There are a few instances noted in the literature of home remedies, and several noted in Dangerous Marine Animals, by Dr. Bruce W. Halstead of the World Life Research Institute that are of interest. Halstead on several occasions refers to using sand or adhesive tape to strip the wound site after a sting. Auerbach recommends the use of shaving cream and a balded razor to strip the outer layers for treatment of biological envenomations. Such an act seems to be clearly counter to the accepted practice for treating such stings.

The basis for the current invention to treat such stings is founded on the concept of preparation of the wound site and delivery of the toxin antagonist. A generally inert material in the form of a pad with an abrasive texture is saturated with a solution specific to the desired action in various instances. The texture of the pad can be seen as being similar to the surface of pads used in the kitchen for cleaning pots and pans. The wound or sting is scrubbed with the pad containing the solution. The key is the application of the solution during the abrading or scrubbing of the wound. The solution that saturates the pad can be a ratio of soap and water that provides sufficient detergence to clean the wound and a a pH desired lavage. Furthermore, the pads can be impregnated with various solutions composed of enzymes, hypo-allergenic soap solutions, antihistamines solutions, anesthetic solutions, antibacterial solutions, antifungal solutions, antibiotic solutions, and combinations of any of the above for the specific treatment desired.

### BRIEF DESCRIPTION OF THE DRAWINGS

FIG. 1 illustrates the length of typical Solenopsis Invicta stinger.

FIG. 2 illustrates the average human epidermal layer of skin.

FIG. 3 illustrates an armed nematocyst

FIG. 4 illustrates a discharged nematocyst.

FIG. 5 illustrates a side projected view of a pad containing a typical solution.

### PREFERRED EMBODIMENTS OF THE INVENTION

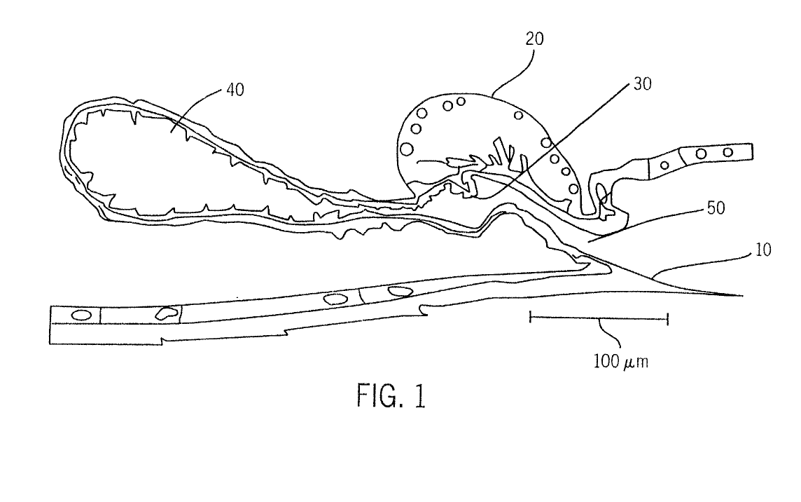

Solenopsis Invicta is a specific group of ants that are internationally known for their aggressive behavior and their extremely painful and dangerous stings. Some of the stings of the Solenopsis Invicta have been fatal. Characteristic of Solenopsis Invicta and a host of other stinging arthropod is that their toxins although powerful are largely deposited in rather shallow wound sites due to the diminutive size of the stinger. As illustrated in FIG. 1, the Stinger 10 has a length of about 100 microns. The glandular epithelium 20 deposits or emits toxin 30 to reservoir 40. The stinger 10 opens a hole or wound opening in the top layer of skin, the epidermis, of a recipient of about ½ to ⅔ of the length of the stinger and the toxin 30 enters the wound opening or hole via passage 50.

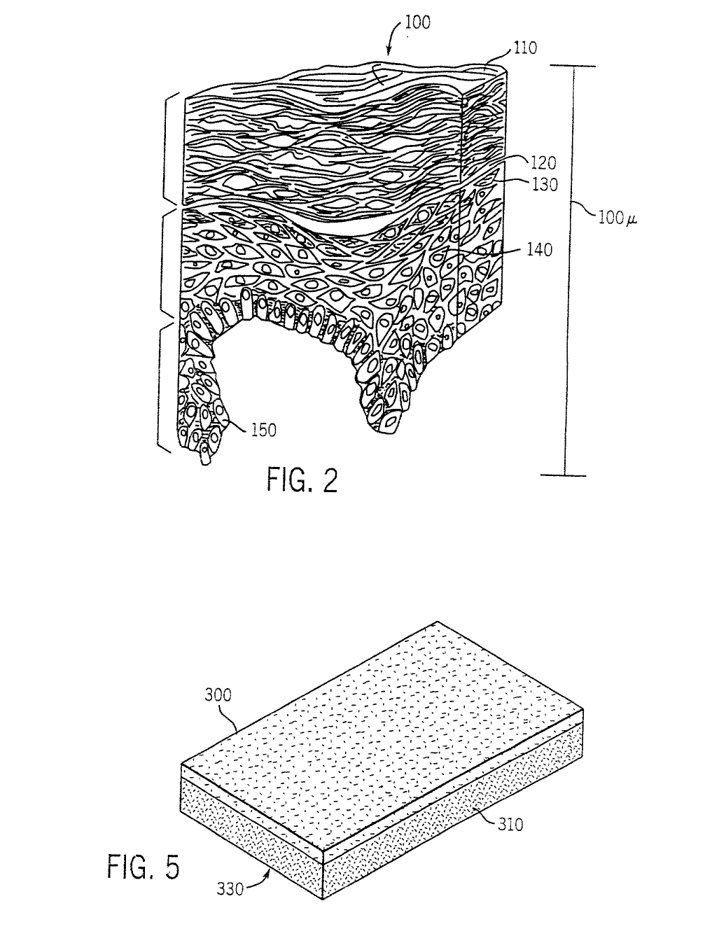

FIG. 2 illustrates the average human epidermis 100 having a typical thickness ranging from 70 to 120 microns or an average thickness of 100 microns. The epidermis 100 has five distinct layers. The first layer 110 being the Stratum corneum. The second layer 120 being the Stratum lucidum. The third layer 130 being the Stratum granulosum. The fourth layer 140 being the Stratum spinosum. And the fifth layer 150 being the Stratum basale.

Physicians treating dermatological disorders have long used demabrasion or “Skin Planing” to treat problems ranging from acne to burns, dead tissue removal, and even cosmetically to eliminate wrinkles. Simply put—sand paper, emery cloth, files, graters and even wire bristles are used to strip the upper portion of the skin or epidermis from the dermis in anticipation of a smooth, non-pathological re-epidemermization of the area (hopefully no more acne or wrinkles).

The use of abrading the epidermis for delivery systems for treatments and medications of the current invention can be analogized to the hypodermic needle; however, as the name indicates, hypo (beneath) dermis (skin) causes a deep wound for delivery or retrieval purposes. With the current invention, the method describes, e.g. delivery of toxin antagonists to shallow level where the toxins were delivered by the Solenopsis Invicta in the original wound or incursion and the instant invention may be thought of as a micro hypo “epi” dermic delivery system or an endodermic delivery system.

Besides the arthropod, the invention is applicable for Cnidarian and other biological envenomations (a.k.a. Coelenterates) stings. The Cnidarians consist of hydras, men of war, jellyfish, sea anemones, hydrodroids, corals, bryozoans and the like. Characteristic of Cnidarians are nematocysts. These are the stinging cells which are usually deployed for food acquisition and/or for defensive purposes. Since all Nematocyst-bearing Cnidarians have essentially the same stinging cells, the Nematocysts of prime concern are stinging and stinging/tangling and will be generally dealt with, rather than specifically what was done with the Solenopsis Invicta. FIGS. 3 and 4 illustrate an armed Nematocyst 200 and a Discharged Nematocyst 210 respectively.

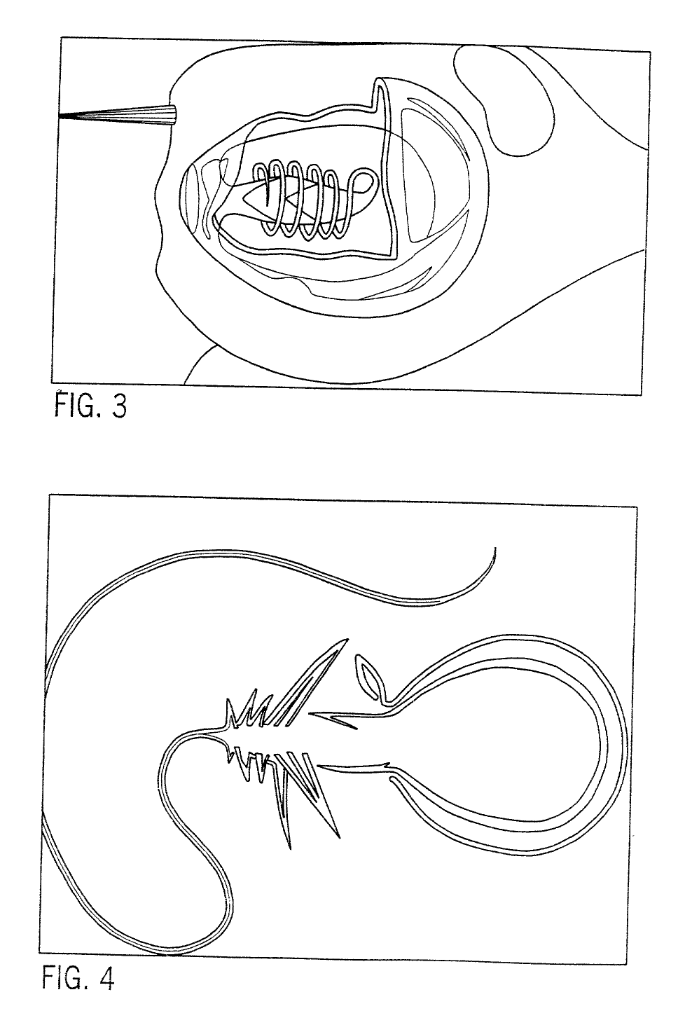

Because the Cnidarians use of a similar diminutive stingers size as with the Solenopsis Invicta, the use of the instant invention is similarly effective for the same reasons. The abrasive Debridement characteristics of the delivery pad are illustrated in FIG. 5 at 300 having a solution 310 saturated in the pad, 300. In addition, with many of the Cnidarians the nematocysts are barbed and pull away with the victim. The abrasive Debridement characteristics of the delivery pad 300 facilitate removal of these otherwise anchored weapons. From side 320 to 330, the pad can go from very abrasive to less abrasive, or even abrasive to smooth from one side to another. The pad could also be multilayered with a absorbent material backing a porous abrasive material where the absorbent material would carry the solution and be delivered through the abrasive material during application of slight pressure. An alternative could also be a type of bladder alone or in combination with the abrasive and absorbent material where the abrasive side would abrade and the application of pressure would burst the bladder releasing the solution at the desired moment or saturating the absorbent side thereby have at least a three layer pad with the bladder between the abrasive side and the absorbent side of the pad. The delivery of multiple combination could take place with multiple bladders of different solutions being stacked up with the abrasive material so that the mixing of different solution could take place at the desired time or in desired sequence with the bladders being of different sequencing for bursting for specific combinative application of a solution.

The instant invention was tested on a number of humans, approximately one hundred or more individuals, that had received stings of the type disclosed above. The recipient or victims took the a similar pad as described above that was saturated with a solution of soap and sterile water and a 2% Papain Enzyme that was manufactured in a foil type package similar to a towelette. Unlike the towelette, which is durable and soft, the instant pad is somewhat stiff and has an abrasive texture sufficient to plane the skin and deliver the solution to the planed epidermis. The method of the instant invention was used with success by over ninety-five percent of the people with immediate resolution of suffering by the recipient. The instant invention was also successful on bites or stings of unidentified species and genus.

During the discovery and clinical testing period, variation on the original method and pad became evident. Since demabrasion pad is premier delivery system for antitoxin, materials as described above for these series of pests, the treatment for use against other marine and land pests as listed in Tables 1 and 2 below have been accomplished and should be treated successfully with instant invention because the delivery of a toxin is similar as the instances above and the delivery of the antitoxins would be expected to have the same result.

Further, the medication that will be saturated into the abrading paid will be in the form a solution of a sufficient dilution to permit transmission from the abrading pad during abrading or treatment of a wound or the surface of the recipient's skin. The solution in the pad can be a simple sterile water with a mild soap, an antibacterial formulation, a combination of water and isopropyl alcohol, water and hydrogen peroxide, an iodine solution and similar solution for cleaning and disinfecting or preventing infection.

Alternatively, the abrasive material can be a paste or a gel of sufficient abrasive character to abrade the epidermis sufficient to deliver the solution. The solution can be part of the constituents of the gel or paste, or separable therefrom through the use of a pad of material that would carry the solution.

Additionally, it is anticipated that this method of delivery for antitoxins for pests can be used as a delivery system for other dermatological medications, chemicals and variations combination of other medications and solutions, such as a prescription drug for delivery just below the surface of the skin, or an over the counter analgesic or skin treatment.

Not only may one consider using the instant invention for demabrasion and debridement but the cleansing that occurs with a lightly abrasive pad accompanied with a detergent could lend itself to a variety of other application particularly used in conjunction with a combination of other ingredients. Transdermally dispensed medications such as scopolamine, atropine and nicotine preparations would have their efficacy enhanced by lightly abrading the medication site, as well as all other topical and transdermal prescription medications.

As noted the solution that saturates the pad can be a ratio of soap and water that provides sufficient detergence to clean the wound and a pH desired lavage. Furthermore, the pads can be impregnated with various solutions composed of enzymes, hypo-allergenic soap solutions, antihistamines solutions, anesthetic solutions, antibacterial solutions, antifungal solutions, antibiotic solutions, and combinations of any of the above for the specific treatment desired.

The selection of a construction or materials are not limited to those disclosed because the disclosure is one of example and not limitation. The abrasive material can be made of natural fibers such as linen, flax, cotton or wool, or plastic, or metal, or paper or a combination of all or some of the materials. The pad itself alone or the use with the gel or paste can be made of natural fibers such as linen, flax, cotton or wool, or plastic, or metal, or paper or a combination of all or some of the materials being of varying degrees of abrasiveness or smoothness. Furthermore, the present invention can incorporate just an abrasive pad saturated with a solution, or it can be a pad with a varying degree of abrasiveness on one side very abrasive and on the other side less abrasive, with the varying of degrees from side to side going from very abrasive on one side to almost smooth on the other side. While the present invention has been described with respect to what is presently an preferred embodiment and apparatus practicing the instant invention, it is understood that the invention is not limited to the disclosed embodiments or examples. To the contrary, the present invention is intended to cover various modifications and equivalent arrangement and configurations and steps included within the spirit and scope of the appended claims. The scope of the following claims is to be accorded the broadest interpretation so as to encompass all such modifications and equivalent arrangements and functions.

TABLE 1

| CLASSIFICATION | COMMON NAME | DISTRIBUTION |
|---|---|---|
| PORIFERA | | |
| Family Desmacidonidae | Red Moss Sponge | U.S. Cape Code to |
| Family Tendaniidae | Fire Sponge | S. Carolina West |
| | | Indies |
| COELENTERATA | | |
| Hydrozoans | | |
| Family Milliporidae | Stinging and/or Fire | Tropical Pacific and |
| | Coral | Indian Oceans, Red |
| | | Sea, & Carribean |
| Family Plumulariidae | Stinging Seaweed | Tropical Pacific & |
| | | Indian Oceans. West |
| | | Indies |
| | Atlantic Portuguese | Atlantic Ocean & |
| | Man-o-War | Mediterranean Sea |
| Family Physaciidac | Indo-Pacific | Tropical Indian & |
| | Portuguese | Pacific Oceans |
| | Man-o-Ware | |
| SCYPHOZOA | | |
| *Family Chirodropidae¹ | Deadly Sea Wasp: | Australian Pacific |
| | Box Jelly | Coast |
| Family Carbdeidae | Sea Wasp | Tropical Pacific, |
| | | Atlantic & Indian |
| | | Oceans |
| Family Chirodropidae | Sea Wasp | Tropical Pacific, |
| | | Atlantic & Indian |
| | | Oceans |
| Family Cyaneidae | Sea Bubber; Lion's | Northern Atlantic & |
| | Mane | Pacific, Baltic Sea, |
| | | Tropical & Temperate |
| | | Pacific Ocean |
| ANTHOZOA | Sea Anemones & | World-wide |
| | Coral | |
| Family Acroporidae | Elkhorn Coral | Caribbean Sea |
| Family Actiniidae | Sea Anemone | Eastern Atlantic, |
| | | Mediterranean, Black |
| | | Sea & Sea of Asia |
| Family | Hell's Fire Sea | Atlantic & Pacific |
| Actinodendronidae | Anemone | Oceans |
| Family Aliciidae | Sea Anemone | Red Sea |
| Family Hormathiidae | Sea Anemone | Eastern Atlantic, |
| | | Mediterranean Sea, |
| | | Coastal Africa |
| ANNELIDA | Segmented Worms | World-wide |
| Family Anphinomidae | Bristle Worm | Gulf of Mexico, |
| | | Tropical Pacific & |
| | | Atlantic Oceans |
| ENCHINCDERMATA | Starfish, Sea | World-wide |
| | Cucumbers, Sea | |
| | Urchins | |
| Class Hoeothuriodea | Sea Cucumber | All Temperate Bodies |
| | | of Water |
| Family Toxopreustidae | Sea Urchin | Pacific & Indian |
| | | Oceans |
| ECTOPEOCTA | | |
| Family Acyonidiidae | Bryozanns | World-wide |

¹*Efficacy her is theorized & not tested

TABLE 2

| ARTHROPODA | Spiders and Insects | Global |
|---|---|---|
| Myrmecinae | Ants | World-wide |
| Examples: | | |
| SolStingingenopsis | Fire Ants | World-wide |
| Pogonomyemex | And | |
| Paraponera | Other Ants | |
| Diptera and Acarina | No-see-ums, punkies | World-wide |
| Nematocera | gnats, moose flies. | |
| Ceratopogonidae | midges, biting midges | |
| Culicoides | blood sucking gnats, | |
| Acari | mites, chigoe, | |
| Culex | jigger, chigger, fleas. | |
| Trichoceridac | blow flies, blue bottle, | |
| Mycelophilidae | biting housefly, | |
| Chaoborinae | stable fly, | |
| Calliphoridae | buffalo gnats, | |
| Sinuliidae | black flies | |

What is claimed is:

1. A method for delivering a sub-epidermal treatment, comprising the steps of:
a. preparing a medicating solution;
b. saturating a saturatable material with sufficient abrasive characteristics to abrade the epidermis; and
c. abrading the epidermis with the saturatable material, wherein the abrasion enables sub-epidermal delivery of the medicating solution in the saturatable material.

2. The method of claim 1, wherein the medicating solution comprises soap and water.

3. The method of claim 1, wherein the medicating solution contains an antitoxin.

4. The method of claim 1, wherein the sub-epidermal treatment is for treating a wound.

5. The method of claim 4, wherein the wound has been caused by an arthropod.

6. The method of claim 5, wherein the arthropod is Solenopsis Invicta.

7. The method of claim 4, wherein the wound was caused by a Cnidarian.

8. The method of claim 7, wherein the Cnidarian is a larval of the thimble jellyfish.

9. The method of claim 1, wherein the medicating solution contains an antibiotic.

10. The method of claim 4, wherein the wound was biologically induced.

11. The method of claim 1, wherein the medicating solution contains an antifungal treatment.

12. The method of claim 1, wherein the medicating solution contains an antiseptic.

13. The method of claim 1, wherein the solution is a pH titer.

14. The method of claim 1, wherein the treatment is directed towards an intended surgical site.

15. The method of claim 1, wherein the treatment is directed towards a lesion.

16. The method of claim 15, wherein the lesion is an ulcer.

17. The method of claim 1, wherein the saturatable material with sufficient abrasive characteristics to abrade the epidermis is a paste, and wherein the paste has a sufficient grittiness to abrade the epidermis.

18. A method for treating a biological envenomation, comprising the steps of:
a. preparing a medication in the form of a solution;
b. saturating a saturatable material with sufficient abrasive characteristics to abrade the epidermis; and
c. abrading the epidermis transdermally to a sub-epidermal level where a wound has occurred with the saturatable material.

19. The method of claim 18, wherein the medicating solution comprises soap and water.

20. The method of claim 1 9, wherein the medicating solution contains an antitoxin.

21. The method of claim 21, wherein the sub-epidermal treatment is for treating a wound.

22. The method of claim 21, wherein the wound has been caused by an arthropod.

23. The method of claim 22, wherein the arthropod is Solenopsis Invicta.

24. The method of claim 21, wherein the wound was caused by a Cnidarian.

25. The method of claim 24, wherein the Cnidarian is a larval of the thimble jellyfish.

26. The method of claim 1 8, wherein the medicating solution contains an antibiotic.

27. The method of claim 1 8, wherein the medicating solution contains an antifungal treatment.

28. The method of claim 1 8, wherein said solution is a pH titer.

29. The method of claim 1 8, wherein the treatment is directed towards an intended surgical site.

30. The method of claim 1 8, wherein the treatment is directed towards a lesion.

31. The method of claim 30, wherein the lesion is an ulcer.

32. The method of claim 1 8, wherein the saturatable material with sufficient abrasive characteristics to abrade the epidermis is a paste, and wherein the paste has a sufficient grittiness to abrade the epidermis.

33. A method for delivering a sub-epidermal treatment, comprising the steps of:
a. preparing a medicating solution;
b. saturating a saturatable material with sufficient abrasive characteristics to abrade the epidermis; and
c. abrading the epidermis with the saturatable material transdermally to a sub-epidermal level,, wherein the abrasion enables sub-epidermal delivery of the medicating solution in the saturatable material.

34. The method of claim 33, wherein the medicating solution comprises soap and water.

35. The method of claim 34, wherein the medicating solution contains an antitoxin.

36. The method of claim 35, wherein the sub-epidermal treatment is for treating a wound.

37. The method of claim 36, wherein the wound has been caused by an arthropod.

38. The method of claim 37, wherein the arthropid is Solenopsis Invicta.

39. The method of claim 36, wherein the wound was caused by a Cnidarian.

40. The method of claim 39, wherein the Cnidarian is a larval of the thimble jellyfish.

41. The method of claim 33, wherein the medicating solution contains an antibiotic.

42. The method of claim 33, wherein the medicating solution contains an antifungal treatment.

43. The method of claim 33, wherein said solution is a pH titer.

44. The method of claim 33, wherein the treatment is directed towards an intended surgical site.

45. The method of claim 33, wherein the treatment is directed towards a lesion.

46. The method of claim 45, wherein the lesion is an ulcer.

47. The method of claim 33, wherein the saturatable material with sufficient abrasive characteristics to abrade the epidermis is a paste, and wherein the paste has a sufficient grittiness to abrade the epidermis.

48. A method for improved transdermal delivery of a medicating solution, comprising the steps of:
a. preparing a medicating solution;
b. saturating a saturatable material with sufficient abrasive characteristics to abrade the epidermis; and
c. prior to application of a transdermal delivery system, abrading the epidermis where the transdermal system will contact the surface of the component of a recipient.

49. The method of claim 48, wherein the medicating solution is of a sufficient dilution to permit transmission from the abrading pad during abrading or treatment of a wound or the surface of the skin of the recipient.

50. The product for practicing the method of claim 1, comprising:
an abrasive material;
wherein said abrasive material is capable of abrading at least part of the epidermis; and
wherein said abrasive material is capable of being saturated with a medicating solution.

51. The product for practicing the method of claim 18, comprising:
an abrasive material;
wherein said abrasive material is capable of abrading at least part of the epidermis; and
wherein said abrasive material is capable of being saturated with a medicating solution.

52. The product for practicing the method of claim 33, comprising:
an abrasive material;
wherein said abrasive material is capable of abrading at least part of the epidermis; and
wherein said abrasive material is capable of being saturated with a medicating solution.

53. A method for delivering a sub-epidermal treatment, comprising the steps of:
a. preparing a medicating solution;
b. saturating a saturatable material with sufficient abrasive characteristics to abrade the epidermis, wherein the saturatable material with sufficient abrasive characteristics to abrade the epidermis is a paste; and
c. abrading the epidermis wherein the abrasion enables sub-epidermal delivery of the medicating solution in said saturatable material.

54. The method of claim 53, wherein material with sufficient abrasive characteristics to abrade the epidermis is a gel with sufficient abrasive characteristics to abrade the epidermis.
